(19) United States
(12) Patent Application Publication
(10) Pub. No.: US 2004/0161763 A1
(43) Pub. Date: Aug. 19, 2004

(54) Fluroscence polarisation

(76) Inventors: Kurt Berlin, Stahnsdorf (DE); Jurgen Distler, Berlin (DE)

Correspondence Address: Kriegsman & Kriegsman, 665 Franklin Street, Framingham, MA, 01702, US

(21) Appl. No.: 10/470,695

(22) PCT Filed: Jan. 29, 2002
(86) PCT No.: PCT/EP02/00923

(30) Foreign Application Priority Data
Jan. 29, 2001 (DE) 101 04 938.2

(51) Int. Cl. C12Q 1/68

(52) U.S. Cl. 435/6

(57) ABSTRACT

A method for the analysis of the methylation of cytosine bases in genomic DNA samples, comprising the following steps: (a) the genomic DNA is chemically treated in such a manner that cytosine is converted into uracil or a similar base regarding the base pairing behaviour in the DNA duplex, 5 methylcytosine however remains unchanged; (b) the chemically treated DNA is amplified using of at least one species of oligonucleotide (type A) as a primer in a polymerase reaction; (c) the amplificate is left in solution with one or more species of fluorophore labelled nucleotides and one or more species of oligonucleotide (type B), wherein the type B oligonucleotide hybridises under appropriate conditions with its 3′ end directly on or up to 10 bases from the position to be examined, and wherein said type B oligonucleotide is at least partly nuclease resistant; (d) the hybridised oligonucleotide (type B) is extended by means of a polymerase by at least one nucleotide, whereby the extension is dependent upon the methylation status of the respective cytosine position in the genomic DNA sample; (e) the solution is incubated with a phosphodiesterase, which is capable of digesting nucleic acids, however incompletely digests the type B oligonucleotides and its extension products; (f) the fluorescence polarisation of the solution is measured whereby for each fluorescent label used one determines the degree of polarisation.

### FIELD OF THE INVENTION

This invention relates to a method for the analysis of methylation patterns in genomic DNA, for use in high throughput analysis, research or clinical settings. This method utilises bisulfite treatment and fluorescence polarisation assay techniques.

### BACKGROUND OF THE INVENTION

The levels of observation that have been studied in recent years in molecular biology have concentrated on genes, the translation of those genes into RNA, and the transcription of the RNA into protein. There has been a more limited analysis of the regulatory mechanisms associated with gene control. Gene regulation, for example, at what stage of development of the individual a gene is activated or inhibited, and the tissue specific nature of this regulation is less understood. However, it can be correlated with a high degree of probability to the extent and nature of methylation of the gene or genome. From this observation it is reasonable to infer that pathogenic genetic disorders may be detected from irregular genetic methylation patterns.

### STATE OF THE ART

### Methylation and Disease

The efforts of the Human Genome project are concentrated on the sequencing of the human genome. It is expected that this will yield considerable therapeutic and diagnostic benefits for the treatment of disease. However, these efforts have so far been unable to address a significant aspect of genetic disorders, the epigenetic factor. The epigenetic regulation of gene transcription has been shown to effect many disorders. One of the most significant epigenetic mechanisms so far identified has been the methylation of cytosine. The methylation of cytosine at the 5 position is the only known modification of genomic DNA. Although the exact mechanisms by which DNA methylation effects DNA transcription are unknown, the relationship between disease and methylation has been well documented. In particular, methylation patterns of CpG islands within regulatory regions of genome appear to be highly tissue specific. Therefore, it follows that misregulation of genes may be predicted by comparing their methylation pattern with phenotypically ‘normal’ expression patterns. The following are cases of disease associated with modified methylation patterns.

Head and neck cancer (Sanchez-Cespedes M et al. “Gene promoter hypermethylation in tumours and serum of head and neck cancer patients” Cancer Res. 2000 Feb. 15; 60 (4):892-5)

Hodgkin's disease (Garcia J F et al “Loss of p16 protein expression associated with methylation of the p16INK4A gene is a frequent finding in Hodgkin's disease” Lab invest 1999 December; 79 (12):1453-9)

Gastric cancer (Yanagisawa Y et al. “Methylation of the hMLH1 promoter in familial gastric cancer with microsatellite instability” Int J Cancer 2000 Jan. 1; 85 (1):50-3)

Prader-Willi/Angelman's syndrome (Zeschnigh et al “Imprinted segments in the human genome: different DNA methylation patterns in the Prader Willi/Angelman syndrome region as determined by the genomic sequencing method” Human Mol. Genetics (1997) (6) 3 pp 387-395)

ICF syndrome (Tuck-Muller et al “CMDNA hypomethylation and unusual chromosome instability in cell lines from ICF syndrome patients” Cytogenet Call Genet 2000; 89(1-2):121-8)

Dermatofibroma (Chen T C et al “Dermatofibroma is a clonal proliferative disease” J Cutan Pathol 2000 January; 27 (1):36-9)

Hypertension (Lee S D et al. “Monoclonal endothelial cell proliferation is present in primary but not secondary pulmonary hypertension” J clin Invest 1998 Mar. 1, 101 (5):927-34)

Autism (Klauck S M et al. “Molecular genetic analysis of the. FMR-1 gene in a large collection of autistic patients” Human Genet 1997 August; 100 (2): 224-9)

Fragile X Syndrome (Hornstra I K et al. “High resolution methylation analysis of the FMR1 gene trinucleotide repeat region in fragile X syndrome” Hum Mol Genet 1993 October, 2(10):1659-65)

Huntigton's disease (Ferluga J et al. “possible organ and age related epigenetic factors in Huntington's disease and colorectal carcinoma” Med hypotheses 1989 May; 29(1); 51-4)

All of the above documents are hereby incorporated by reference.

The state of the art covers two basic methods for the analysis of methylation patterns and nucleic acids. The first concerns a method for the analysis of methylation patterns at specific sites in the genome. The second concerns a method that utilises fluorescent polarisation for the analysis of nucleic acids.

### Detection of Cytosine Methylation in DNA

The modification of the genomic base cytosine to 5-methylcytosine represents the epigenetic parameter which to date is the most examined and understood. Nevertheless, the characterisation of this epigenomic parameter is still not on par with that of genotyping of cells and individuals. There is still room for the development of more methodologies for the high throughput analysis and characterisation of the methylation patterns of cells. The most comprehensive patent covering this field, is WO 99/28498, which is hereby incorporated by reference. Said invention providing a means for the detailed analysis of methylation patterns. The disclosed invention aims to provide an alternative solution by utilising fluorescence polarisation techniques in the analysis of methylation patterns. It will provide a simple methylation assay especially suitable for a medium throughput clinical environment.

Standard methods of sequence analysis such as cloning and PCR are insufficient for the analysis of methylation as covalent modifications to the DNA such as methylation are not conserved.

There are currently three methods used for the differentiation of 5-methyl cytosine from unmethylated cytosine in DNA sequence.

The first method uses restriction enzymes. Restriction endonucleases cut DNA sequences at specific locations, upon recognition of a specific sequence, usually 4-8 bases in length. These enzymes are highly specific as to the sequence they recognise. In some cases, known as ‘methylation sensitive’ they will not cut at the methylated version of the recognition sequence. Therefore methylation sensitive enzymes can be used to identify methylation within restriction enzyme sites.

The position of the cuts may be determined by gel electrophoresis, followed by blotting and hybridisation. This method has not proved useful for the efficient identification of methylated CpG sites in the genome for two reasons. Firstly, most CpG islands that are methylated are not within the recognition sequence of most restriction enzymes. Secondly, the sensitivity of this method is extremely low (Bird, A. P., Southern, E. M., J. Mol. Biol. 118, 27-47). The sensitivity can be improved by amplifying the region after restriction exonuclease digestion. Two primers are used that flank the recognition site of the enzyme. In the event of the digestion taking place amplification will not occur. The amplification products can then be analysed by blotting and hybridisation to identify the site of the cut. In theory the resolution of this technique can be one base pair. However, as it is highly labour intensive and costly it is not a practical solution to the large scale analysis of methylation patterns. (Shemer, R. Et al., PNAS 93, 6371-6376)

The second method utilises the sequencing method developed by Maxam Gilbert, for 5-methyl cytosine identification. The technique involves the partial chemical cleavage of whole DNA followed by ligation, amplification and hybridisation. In theory regions having a size of less than 1000 base pairs can be analysed. However, this method is so complicated and unreliable that it is rarely used.

### Bisulfite Treatment

The preferred method of methylation analysis involves a chemical modification of the DNA sequence. The method is based on the bisulfite conversion of cytosine to uracil. DNA is denatured and then treated using a bisulfite solution. This results in the conversion of cytosine to uracil, that leaves the methylated cytosines unmodified. Uracil acts as analogue of thymine for base pairing purposes, rather than cytosine. As a result of the bisulfite treatment the DNA strands that were originally complementary to each other, the coding and template strands are no longer complimentary.

Oligonucleotide primers for the amplification of each bisulfite treated strand can then be designed. Enzymatic amplification of the sequence results in the incorporation of thymine nucleotides at positions that were cytosine in the original sequence.

Amplification of the bisulfite treated DNA using bisulfite specific primers results in the formation of a complementary strand, the sequence of which is dependant on the methylation status of the genomic sample, and is thus unique from the original pre bisulfite treated complementary strand. The bisulfite treatment and subsequent amplification therefore results in the formation of 4 unique nucleic acid fragments. These four strands all contain the same information, assuming that methylation has been symmetric, that is, both strands of the CpG position have been methylated. The methylation status of each CpG position may therefore be assessed independently four times.

Current methods for the assessment of the methylation status of a CpG position bisulfite converted sequence include standard chromatographic analysis, hybridisation analysis, or mass spectrometry analysis.

All methods require the purification of the PCR products, for example by gel electrophoresis which may also serve directly for analysis. In the hybridisation analysis the bisulfite treated and PCR amplified nucleic acids are chemically labelled and hybridised to complementary oligonucleotides. The amplified fragments are tested using two labelled oligonucleotides, one which is specific for unmethylated DNA, and therefore is CpG rich, and another specific for methylated DNA which contains no CpG. The hybridisation is then detected by an assay for the label. This form of analysis may be carried out in the form of a DNA array, allowing high throughput analysis. An alternative method, utilising MALDI mass spectrometer analysis of nucleic acids has been described by Kirpekar F et. al. ‘DNA sequence analysis by MALDI mass spectrometry’ Nucleic Acid Research; 26, 2354-9.

### Fluorescence Assays

The disclosed method provides a new use for an established form of fluorescence assay to provide a novel solution to the problem of analysis of chemically modified methylated genomic DNA sequence.

The use of fluorescence techniques for the analysis of small biomolecules is well known. There are currently four commercially available methods for the closed tube luminescence analysis of enzymatic amplification products. These are the Taqman, Molecular Beacons, LightCycler and Amplifluor assays. All are based on the use of fluorescence resonance energy transfer (FRET). FRET is a form of molecular energy transfer whereby energy is passed between donor and acceptor species. Energy is passed non radiativley between an acceptor molecule and a donor molecule. The donor absorbs a photon and passes this non radiatively to the acceptor molecule, thus causing it to fluoresce. When two fluorophores whose excitation and emission spectra overlap are in close proximity, excitation of one fluorophore will cause it to emit light at wavelengths that are absorbed by and that stimulate the second fluorophore, causing it in turn to fluoresce.

All methods based on FRET are characterised by relatively high signal-to-noise ratios and a good ability to discriminate between positive and negative reactions. However, they are all limited in the sense that either a dual label probe or primer or two separate probes per target have to be used. This seriously complicates probe design and synthesis. In addition, since they all employ labels with rapidly decaying fluorescence and broad emission peaks, the possibilities for multiplex detection are limited. The invention proposes the use of fluorescence polarisation as opposed to FRET.

### Fluorescence Polarisation

Most fluorescence assays utilise the fluorescence transfer properties of donor and acceptor groups to observe the properties of small biomolecules. The use of fluorescence polarisation techniques was, until recently, limited to smaller analytes in the region of a molecular weight of about 1,000 Daltons. It had been utilised mainly in a number of immunoassays and for the measurement of microviscosity and molecular volume. One of the main advantages of fluorescence polarisation techniques over other methods is that it allows the analysis of homogenous solutions, i.e. there is no need for purification procedures.

The concept of fluorescence polarisation has been known since the 1920s. It is a measure of the time-average rotational motion of fluorescent molecules.

The fluorescence polarisation technique allows the observation of changes in the rotational properties of molecules in a solution. Molecules in solution rotate and tumble about multiple axis. Fluorescence polarisation relies on the property of plane polarised light to be emitted by a stationary fluorescent molecule. If plane polarised light is used to irradiate a fluorescent molecule, the molecule will emit plane polarised light between excitation and emission only when stationary. Larger molecules, i.e. those of larger molecular weight and/or volume tumble more slowly about their axes than smaller molecules. As the degree of polarisation of the light emitted by the fluorescent molecule is related to the degree of movement of the molecule, it is possible to distinguish between larger and smaller molecules based on the degree of polarisation of light.

In fluorescence polarisation techniques, the fluorescent molecule is first excited by polarised light. The polarisation of the emission is measured by measuring the relative intensities of emission (i) parallel to the plane of polarised excitation light and (ii) perpendicular to the plane of polarised excitation light. A change in the rate of tumbling due to a change in size and/or rigidity is accompanied by a change in the relationship between the plane of excitation light and the plane of emitted fluorescence, i.e., a change in fluorescence polarisation. The observed FP of a species is described by the Perrin equation and is related to the ratio of the rotational relaxation time and the fluorescent lifetime.

Fluorescence polarisation (hereafter referred to as FP) is expressed as a ratio of polarised to non polarised light. As such, it has a distinct advantage over other forms of fluorescence detection in that it is independent of the initial concentration of fluorescence in the solution. As long as the amount of fluorescence is still significantly detectable accurate results can be given. The FP difference between totally bound and totally unbound DNA represents the complete dynamic range of FP. As long as a statistically significant difference can be derived from the interaction of low molecular fluorophore labelled nucleotides and those incorporated into larger nucleic acid molecules FP can be a suitable method for the detection of chemical interactions. However, due to the effects of the local motion of fluorophores it may not always possible to predict the values for reactions, and they may require to be empirically derived.

For a system in which a fluorophore is attached to a nucleic acid of low molecular weight or volume, and is then incorporated into an oligonucleotide primer hybridised to a larger nucleic acid the observed fluorescence (P) may be described as follows:

P=Pₘₐₓ[NTP]b+Pₘᵢₙ([NTP]i−[NTP]b)

where Pₘₐₓ is the polarisation observed for fluorescence labelled NTPs that have been incorporated into the oligonucleotide primer. Pₘᵢₙ is the polarisation observed from the unincorporated dye labelled dNTPs, where [NTP]i is the initial concentration of fluorescent dye labelled dNTPs and [NTP]b is the concentration of incorporated dye labelled dNTP.

It is to be understood that fluorescence polarisation includes all methods of analysis of polarised light emitted from a fluorophore group attached to a dNTP or combined in polynucleotide group. This is state of the art and is described by M. E. Jolley, J. Analytical Toxicology 1981 (5) 236-240 which is hereby incorporated for reference.

The application of FP techniques to nucleic acid analysis was disclosed in patent application EP0382433B1, which is hereby incorporated for reference. The use of FP for nucleic acid sequence analysis has been disclosed in patent publications WO 92/18650 and WO 00/11220, which are hereby incorporated for reference and is known in the state of the art. However, the use of fluorescence polarisation as a tool for the analysis of DNA methylation patterns is unknown. The problem of the invention lies in the analysis of this specialised form of nucleic acid sequence. The methods of analysis currently only possible using chromatography, hybridisation and MALDI mass spectrometer techniques.

### SUMMARY OF THE INVENTION

The invention is a method for the detection of DNA methylation patterns. The state of the art consists of several methods for the analysis of bisulfite converted genomic sequence. However, all entail a two step procedure whereby the bisulfite conversion is followed by a PCR amplification and a subsequent analysis. All current methods of analysis require the purification of nucleic acid products after enzymatic amplification, usually by some form of gel electrophoresis. The present invention provides a significant improvement of the state of the art in that bisulfite sequence analysis may be carried out in a homogenous solution. This allows analysis of the sequence in a closed tube, i.e. concurrent with or upon completion of the enzymatic amplification without need for further purification. In addition the method of the invention may be adapted to other diagnostic formats, for example, high density DNA chip analysis. The method of the invention provides a cost effective method of analysis. Results are obtainable minutes after carrying out the methylation specific reaction.

The proposed invention provides an innovative solution to the problem by providing a novel method comprising the following steps:

a) treatment of nucleic acid sample with a chemical solution in order to convert unmethylated cytosine to uracil.

b) amplifying said treated nucleic acid using oligonucleotide primers specific for the converted sequence

c) hybridising said amplificate with oligonucleotide primers

d) extending said primers by means of fluorophore labelled oligonucleotide probes and polymerase

e) digesting the reaction solution with a phosphodiesterase

f) detecting the fluorescence polarisation of the labelled nucleotides

According to the invention a method for the analysis of the methylation of cytosine bases in genomic DNA samples is provided, comprising the following steps:

(a) the genomic DNA is chemically treated in such a manner that cytosine is converted into uracil or a similar base regarding the base pairing behaviour in the DNA duplex, 5 methylcytosine however remains un-changed;

(b) the chemically treated DNA is amplified using of at least one species of oligonucleotide (type A) as a primer in a polymerase reaction;

(c) the amplificate is left in solution with one or more species of fluorophore labelled nucleotides and one or more species of oligonucleotide (type B), wherein the type B oligonucleotide hybridises under appropriate conditions with its 3′ end directly on or up to 10 bases from the position to be examined, and wherein said type B oligonucleotide is at least partly nuclease resistant;

(d) the hybridised oligonucleotide (type B) is ex-tended by means of a polymerase by at least one nu-cleotide, whereby the extension is dependant upon the methylation status of the respective cytosine posi-tion in the genomic DNA sample;

(e) the solution is incubated with a phosphodi-esterase, which is capable of digesting nucleic ac-ids, however incompletely digests the type B oligonu-cleotides and its extension products;

(f) the fluorescence polarisation of the solution is measured whereby for each fluorescent label used one determines the degree of polarisation.

According to the invention it is preferred, that all or a variable proportion of the fluorophore labelled nucleotides are dideoxynucleotides.

It is further preferred that the polymerase amplification of the bisulfite DNA the nucleotides of the polymerase reaction are diminished by means of a phosphatase and the phosphatase is sub-sequently thermally denatured.

It is also preferred according to the invention that the fluorescence polarisation of the fluorophore labelled nucleotides and/or dideoxynucleotides is measured prior to incorporation into the DNA duplex and again after incorporation into the DNA duplex. It is herein especially preferred that the primer ex-tension is detected by an increase in fluorescence polarisation.

It is also preferred according to the present invention that said fluorophore is selected from the group consisting of 5′carboxyfluorescein, 6-carboxy-X-rhodamine, N,N,N′,N′,-tetramethyl-6-carboxy-X-rhodamine, BODIPY, Texas Red, Cy3, Cy5, FITC, DAPI, HEX, and TET.

It is also preferred that the DNA sample is cleaved prior to bisulfite treatment with restriction endonucleases.

It is especially preferred according to the invention that the DNA sample is isolated from human sources e.g. cell lines, blood, sputum, faeces, urine, brain, cerebrospinal fluid, tissue embedded in paraffin, for example tissue of eyes, intestine, kidney, brain, heart, prostate, lung, chest or liver, histological slides and all possible combinations.

It is also preferred according to the invention that the fluorescence polarisation of the enzymatically amplified DNA is measured directly from the container in which the polymerase reaction was carried out.

According to the invention it is also preferred, that th Type B primers are immobilised on a surface prior to hybridisation with the amplificate.

According to the invention it is also preferred that the bi-sulfite treated DNA is immobilised on a surface prior to hybridisation with the fluorophore labelled nucleotides. It is especially preferred herein, that the surface comprises silicon, glass, polystyrene, alu-minium, steel, iron, copper, nickel, silver or gold.

It is also preferred according to the invention that the information generated about the methylation status at th target site is provided to a computing device comprising one or more databases.

According to the invention it is alos preferred that the in-formation generated about the methylation status at the target site is provided to a computing device comprising one or more learning algorithms.

Another object of the invention is a diagnostic kit comprising:

a) one or more oligonucleotide primers designed to hybridise to bisulphite treated DNA sequence within 1-10 bases 3′ of the target site;

b) at least one species of nucleotides, wherein each species of nucleotide is covalently linked to a unique fluorophore;

c) DNA polymerase that reacts with the oligonu-cleotide primer and nucleotides to produce a 3′ ex-tension of the primer.

According to the invention a kit is preferred, whereby all or a variable pro-portion of the fluorophore linked nucleotides are in the form of dideoxynucleotides.

### DETAILED DESCRIPTION

The methodology consists of the following steps: Firstly the genomic DNA sample must be isolated from tissue or cellular sources. For mammals, more preferaby humans, the DNA sample may be taken from any tissue suspected of expressing the target site within the genome. For mammals, more preferably humans, such sources may include cell lines, blood, sputum, faeces, urine, cerebrospinal fluid, tissue embedded in paraffin; for example, tissue of eyes, intestine, kidney, brain, heart, prostate, lung, chest or liver, histological slides. Extraction may be by means that are standard to one skilled in the art, these include the use of detergent lysates, sonification and vortexing with glass beads. However, in a preferred embodiment the extraction will take place in a minute volume of oil, in order to minimise DNA loss. Once the nucleic acids have been extracted the genomic double stranded DNA is used for analysis.

In a preferred embodiment the DNA may be cleaved prior to the chemical treatment, this may be any means standard in the state of the art, in particular with restriction endonucleases. Said nucleases may include cytosine in the 5′-CpG-3′ context in their recognition sequence, such that the DNA is cleaved only when the cytosines in the recognition sequence are in the unmethylated form.

In a further preferred embodiment the resulting cut ends of the cleaved DNA may be ligated to short double stranded nucleic acid sequences. Said sequences, hereafter known as ‘adaptors’, may present single stranded projections. The adaptors may be attached, for example, by means of a thermolabile ligase enzyme, such as T4 DNA ligase. The ligase is then heat denatured prior to chemical modification of the DNA sample. The adaptors may be of such sequence that they remain unmodified by the chemical treatment used to distinguish methylated from unmethylated DNA sequence. Said adaptors may be used for the enzymatic amplification of the DNA sample by providing a target for the hybridisation of oligonucleotide primers. The use of adaptor molecules is well known within the prior art and will not be elaborated upon.

The sample DNA is then treated chemically in order to convert the methylated cytosine bases into uracil. The chemical modification may be by means of, for example, (but not limited to) a bisulfite solution. Said chemical conversion may take place in any format standard in the the art. This includes but is not limited to modification within agarose gel or in denaturing solvents.

Wherein the chemical modification takes the form of a bisulfite treatment of the DNA the following steps may be followed.

The double stranded DNA must be denatured. This may take the form of a heat denaturation carried out at variable temperatures. For high molecular weight DNA, the denaturation temperature is generally greater than 90 oC. However, the analysis may be upon smaller fragments which do not require such high temperatures. In addition as the reaction proceeds and the cytosine residues are converted to uracil the complementarity between the strands decreases. Therefore, a cyclic reaction protocol may consist of variable denaturation temperatures.

The bisulfite conversion then consists of two important steps, the sulfonation of the cytosine and the subsequent deamination. The equilibra of the reaction are on the correct side at two different temperatures for each stage of the reaction. Taking into account the kinetics of the reactions it is preferable that the reaction takes place under cyclic conditions, with changing temperatures. The temperatures and length at which each stage is carried out may be varied according to the specific requirement of the situation. However, a preferred variant of the method comprises a change of temperature from 4 C (10 minutes) to 50 C (20 minutes). This form of bisulfite treatment is state of the art with reference to WO 99/28498.

Said chemical conversion may take place in any format standard in the the art. This includes but is not limited to modification within agarose gel, in denaturing solvents or within capillaries.

Bisulfite conversion within agarose gel is state of the art and has been described by Olek et al, Nucl. Acids. Res. 1996, 24, 5064-5066. The DNA fragment is embedded in agarose gel and the conversion of cytosine to uracil takes place with hydrogensulfite and a radical scavenger. The DNA may then be amplified without need for further purification steps.

In a further preferred embodiment the DNA conversion may take place without an agarose matrix. The DNA may incubated at increased temperatures with hydrogensulfite and a radical scavenger. Said reaction takes place within an organic denaturing solvent. Examples of denaturing solvents include, but are not limited to, Polyethylene glycol dialkyl polyethylenglycoldialkylether, dioxane and substituted derivatives, urea or derivatives, acetonitrile, primary alcohols, secondary alcohols, tertiary alcohols, DMSO or THF.

In a further embodiment, prior to chemical treatment the DNA sample is transferred into a heatable capillary that is permeable to small molecules. The reaction steps of the chemical modification may then be carried out in the capillary tubes by means of the addition and removal of reagents through connected capillaries.

Subsequent to the chemical treatment the two strands of the DNA may no longer be complementary.

Fractions of the so treated genomic DNA are then enzymatically amplified using oligonucleotide primers. These oligonucleotides which, for example, may be complementary to the adaptor molecules, are hereafter distinguished as type A primers. The length and design of said primers may be specific to the area of the genome to be analysed. As such a wide range of primers are suitable for use in this technique. Such primer design is within the state of the art. The amplification may be such that one strand of the double strands is preferentially amplified, i.e. that one strand is amplified in greater amount than the other.

The amplified DNA solution is then treated with thermolabile enzymes. Excess dNTPs are digested using a phosphatase e.g. shrimp alkaline phosphatase. The enzyme is then denatured using a heat treatment.

The skill of the invention lies in the analysis of the bisulfite treated DNA. In other forms of methylation analysis a purification step is required before further analysis of the methylation patterns can occur. However, one of the advantages of the invention is that the bisulfite treated DNA amplification products may be left in solution.

In one embodiment, the present invention relates to a method for the detection of methylated positions within cytosine rich nucleic acid samples. In such an embodiment the method comprises the contacting of oligonucleotide primers and nucleotides to the DNA solution. A variable proportion of the nucleotides may be labelled with a fluorescent moeity. The present invention further contemplates the use of several fluorescent species as nucleotide labels, whereby each species is unique and may be observed separately observed using fluorescence polarisation. In a preferred embodiment the concentration of the fluorescently labelled nucleotides is selected to be lower or equal to the estimated target site concentration. The oligonucleotide primer is designed to hybridise between 1-10 bases upstream of the target sequence to be analysed. The primers and sequence may be brought together under conditions conducive to hybridisation. The assessment of suitable hybridisation conditions is within the skill of the art. The primers are then extended using a thermostable DNA polymerase with increased efficiency for dye labelled nucleotides, for example, Ampli Taq. In a preferred embodiment primer extension then takes place from said primer with the fluorescent labelled nucleotides.

Subsequent to the primer extension reaction the reaction solution is treated with a phosphodiesterase, said enzyme digesting DNA in a 5′ to 3′ direction. The digestion is carried out in order to degrade any non specific by products, e.g. Type A primers that have hybridised to the amplificate and been extended by means of the fluorophore labelled nucleotides. The incorporation of fluorophore labelled nucleotides into such by products will result in an increase in fluorescence polarisation, in effect providing false positive results. The Type B oligonucleotides may be designed such that a blocking group such as, but not limited to, a phosphorothioate or methylphosphonates or their alkyl derivatives, is carried on one or more base positions. Therefore, when subjected to the phosphodiesterase, digestion will take place only until the base position which has been blocked. In a preferred embodiment the Type A oligonucleotide primers and their extension products are completely digested.

In a further preferred embodiment the fluorescence polarisation of the fluorescent labelled nucleotides is measured prior to incorporation within the DNA duplex. The fluorescence polarisation of the fluorescent labelled nucleotides is then measured after incorporation into the DNA duplex. An increase in FP correlates to the incorporation of the labelled nucleotides in the primer extension. This method allows the analysis of nucleic acids that may not be amenable to standardisation of conditions.

In a further embodiment the nucleotides may take the form of dideoxynucleotides (ddNTPs). In such an embodiment the incorporation of the dideoxynucleotides nucleotides into the primer extension will terminate the primer extension reaction. In a further preferred embodiment a variable proportion of the nucleotides may be ddNTPs.

It is contemplated that all steps of the reaction should take place in a single container. In a further embodiment of the method the reaction may take place bound to a solid surface.

In a further preferred embodiment said primer extension reaction may be substituted with a polymerase chain reaction. In this embodiment the labelled nucleotides would be incorporated into the amplified sequences and would result in an increase in fluorescence polarisation. In such an embodiment, it may be advantageous that the concentration of labelled nucleotides be in excess of the original target sequence. In such an embodiment the nucleotides may be incorporated during multiple PCR cycles, thus allowing an enhancement of the signal.

In a further embodiment the invention may take the form of a kit. The components of said kit should comprise receptacles for the following in sufficient quantities to carry out the examples:

1) Nucleic acid primer;

2) Fluorophore labelled nucleotides;

3) A DNA polymerase that reacts with the primer, sample and nucleotides to produce a 3′ extension of a polynucleotide;

4) Instructions for use;

5) Reagents for the bisulfite conversion of sample DNA to bisulfite sequence.

The term ‘instructions for use’ should cover a tangible expression describing the reagent concentrations for the assay method, parameters such as the relative amounts of reagents to be combined, maintenance times for reagents/sample mixtures, temperature, buffer conditions and such like.

In a further preferred embodiment a variable proportion of the nucleotides may take the form of dideoxynucleotides.

A wide variety of fluorophores are suitable for use in fluorescence polarisation techniques. The selection of appropriate fluorophores is within the skill of the art. Preferred fluorophores include, but are not limited to, 5′carboxyfluorescein (FAM) 6-carboxy-X-rhodamine (ROX); N,N,N′,N′,-tetramethyl-6-carboxy-X-rhodamine (TMR); BODIPY-Texas Red (BTR), CY5, CY3, FITC, DAPI, HEX, and TET. The attachment of the fluorescent labels to the nucleotides is within the skill of the art. In a preferred embodiment of the invention, the length of the linkers used to attach the fluorophores to the bases of the nucleic acids are kept to a minimum, while achieving maximum rigidity. Short and/or rigid linkers keep the movement of the fluorophore relative to the oligonucleotide to a minimum. This allows an increase in the sensitivity of the assay.

The sensitivity of the assay may be increased by decreasing the rotational motility of the bisulfite treated DNA or the primer by increasing their mass. In a preferred embodiment the increase in mass may be achieved by attaching the amplified DNA to small glass beads, small latex beads, hydrophilic functionalized macromolecules or dentrimers. The attachment of such molecules is described in Patent Application WO0023785, which is hereby incorporated for reference.

In a further preferred embodiment the primers may be immobilised on a surface prior to hybridisation with the bisulfite treated DNA. The surface, or solid phase, may be for example, but not limited to, a bead, microplate well or DNA chip. In a further preferred embodiment other reagents of the reaction such as the polymerase may also be bound to the surface. In such an embodiment all reagents may be localised in a microplate well such that the assay may be performed simply by the addition of appropriate buffers and the bisulfite treated DNA sample.

It is anticipated that the method will be used for the high throughput analysis of genomic DNA samples. Therefore the claims also cover a method for the analysis of data using a computing device. In a preferred embodiment said device may comprise one or more databases. In a further preferred embodiment said device may comprise one or more learning algorithms.

### DESCRIPTION OF DIAGRAMS

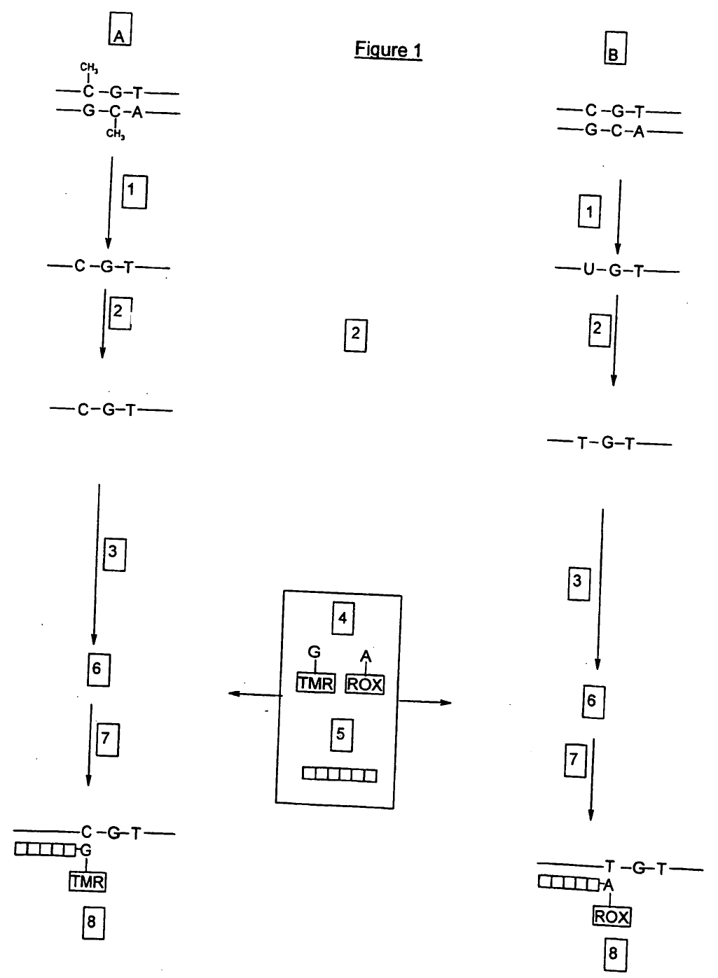

FIG. 1: Incorporation assay

A—Genomic DNA fragment wherein the target sequence is methylated

B—Genomic DNA fragment wherein the target sequence is unmethylated

The genomic DNA is chemically modified such that unmethylated cytosine bases are converted into uracil (1). The target site is amplified by polymerase chain reaction (2). The amplification may be such that only one strand is amplified. Amplified sequence differs from genomic sequence in that methylated cytosine is replaced with thymine, therefore double strands of DNA sequence may no longer be complementary.

The excess nucleotides may then be digested by means of a phosphatase (3). The oligonucleotide primer (5) and dye labelled nucleotides (6) are then contacted with the amplicon. The primer is hybridised with the amplicon at a distance of 1-10 bases from the position to be analysed, and extended using dye labelled nucleotides (7). The reaction solution is digested by means of a phosphodiesterase and the fluorescence polarisation of each label is then measured (8).

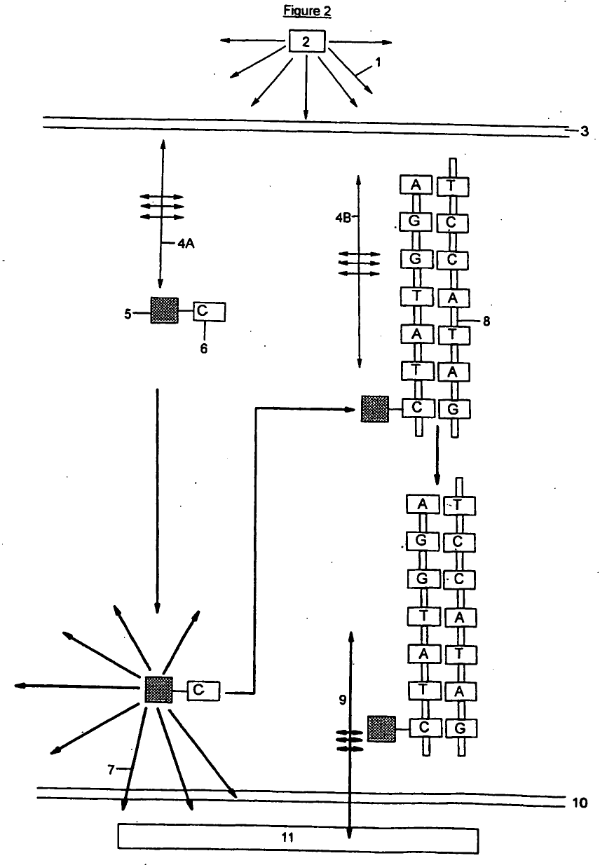

FIG. 2: Measurement of fluorescence polarisation Unpolarised light (1) from a light source (2) is passed though polarisation and colour filters (3). The plane polarised light (4A) is then passed through the reaction solution prior to nucleotide incorporation. The polarised light excites the fluorescent label (5) attached to the nucleotide (6) such that the fluorescent label emits light (7). The nucleotide is free in solution therefore it, and the fluorescent label, have a high degree of motion and emissions are not polarised (7).

The labelled nucleotide is then incorporated into a larger nucleic acid (8). Due to the increase in molecular weight the fluorescent label has a lower degree of motion. Therefore, when excited by the plane polarised light (4B), the emissions (9) have a higher degree of polarisation. The emissions are then passed through polarisation and colour filters (10). The emissions are measured using a fluorimeter (11).

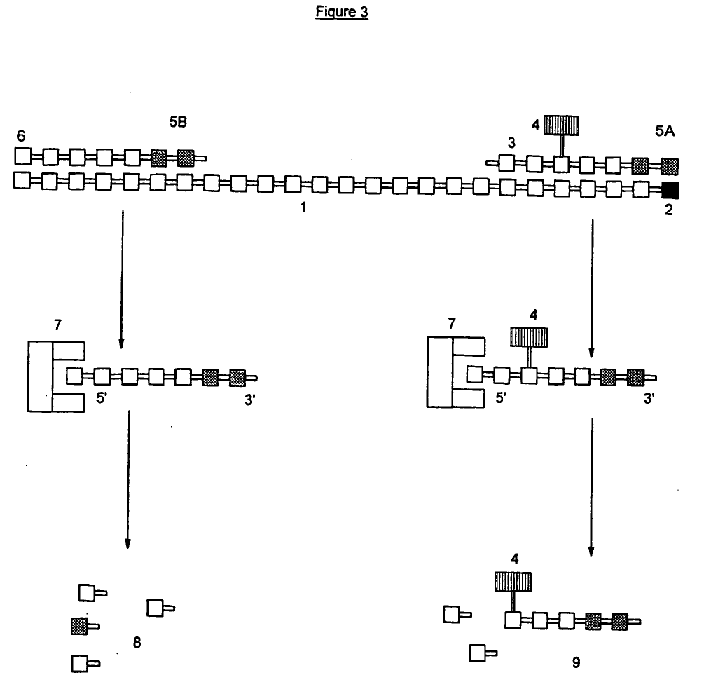

FIG. 3: Phosphodiesterase digestion of by products The amplificate (1), with a target site (2) is hybridised with a Type B oligonucleotide primer (3). The Type B oligonucleotide (3), carries a group (4) that blocks nuclease digestion, and the oligonucleotide (3) is extended by means of fluorescently labelled nucleotides (5A). A Type A oligonucleotide (6) from a previous reaction has also annealed to the amplificate and been extended by means of fluorescently labelled nucleotides (5B), this results in an increase in fluorescence polarisation that is independent of the target site status.

The reaction solution is digested by means of a phosphodiesterase (7) that digests from the 5′ end to the 3′ end. The Type A primer by product is completely digested (8), the fluorescent labelled nucleotides are released into solution and fluorescent polarisation decreases. The Type B oligonucleotide product is only partially digested (9) as complete digestion is blocked by the group (4). As the fluorescent labelled nucleotides are still incorporated into a larger molecule fluorescence polarisation is high.

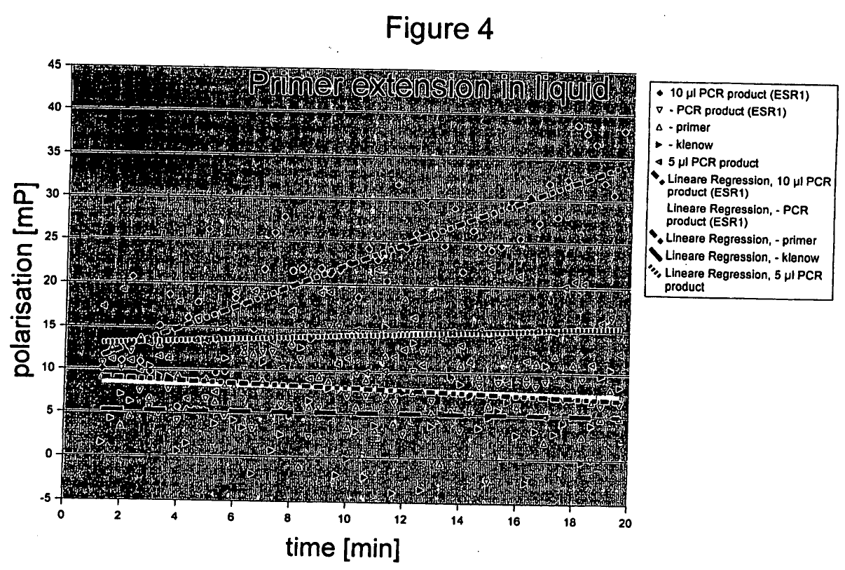

FIG. 4 The diagram shows the increase in fluorescence polarisation of labelled nucleotides when incorporated into nucleic acid as described in Example 1.

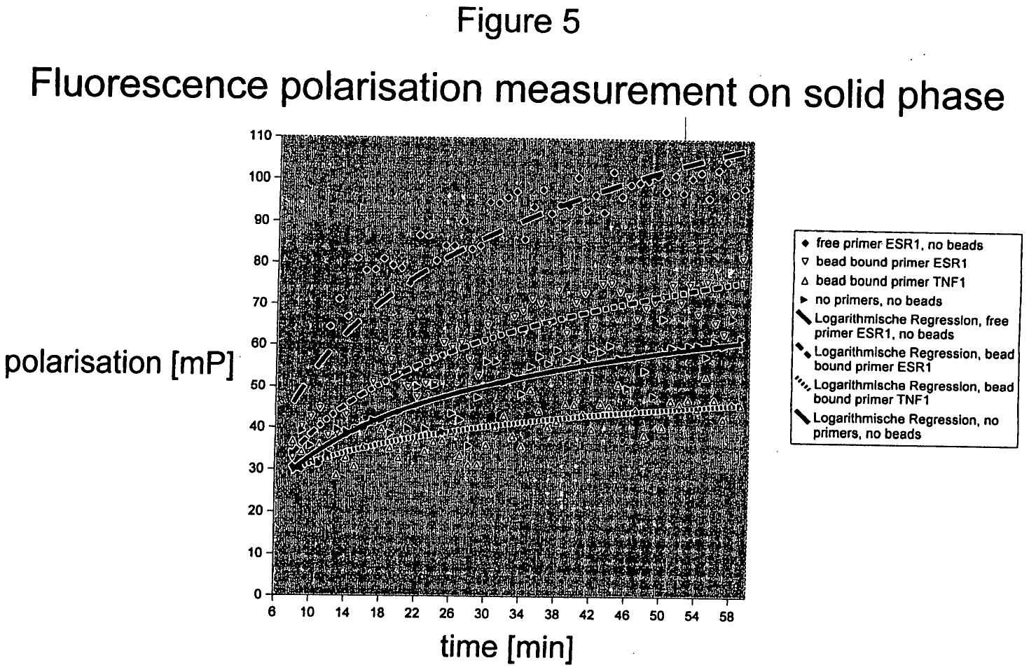

FIG. 5 The diagram shows the increase in fluorescence polarisation of labelled nucleotides when incorporated into nucleic acid bonded to a solid phase as described in Example 1.

### EXAMPLES

### Example 1

In the following example the methylation status of the genes ER1 and TNF were analysed using flourescensce polarisation analysis of flourescently labelled nucleotides incorporated during a primer extension reaction.

In the first step of the reaction double stranded DNA to be analysed was bisulphite treated in order to convert unmethylated cytosine within the sample into thymidine, unmethylated cytosine remaining unaffected by the treatment. The bisulphite treated DNA was subsequently PCR amplified and the purified PCR product was reamplified using asymmetric primer concentrations in order to amplify the G-rich (forward) strand. The single stranded template was then analysed using a primer extension reaction wherein flourescent labelled dATPs were incorporated at cytosine positions which had been unmethylated in the original DNA sample. Incorporation of dATP results in an increase in fluorescence polarisation, therefore the degree of methylation within the DNA sample is inversely proportional to the degree of fluorescence polarisation.

Asymetric PCR Conditions:

1 μL PCR product
0.2 μl Taq
0.2 μl dNTP (25 mM each) 0.2 mM final
1 μl Primer1 AGGAGGGGGAATTAAATAGA
1 μl Primer2 ACAATAAAACCATCCCAAATAC
2.5 μl buffer
19.1 H2O

Program:

95° C./15:00;

10 cycles: 93° C./0:20; 55° C./0:30; 72° C./0:40;

40 cycles: 93° C./0:20; 55° C./0:30; 72° C./0:40+2 sec/cycle

72° C./10:00

4° C./end

The single stranded product was then analysed in a primer extension reaction. In the first instance the experiment was carried out in solution, in the second experiment the experiment was carried out whereby the single stranded amplificate was bound to a solid phase, in this case, beads.

Primer Extension Reaction in Solution

All reactions were carried out within a BMG microplate (black) with a plane bottom. Fluoresceine labelled dATPs were used in the primer extension reaction, incorporation of the dATPs thus indicating the degree of methylation within the original DNA fragment.

A ‘mastermix’ containing water, dNTPs, buffer and dATP-Fluoresceine was prepared, the mix was distributed between the wells of the plate and primer and template DNA added according to the experimental set up. After this gain adjustment of the fluorsecence polarisation instrument (Polarstar Galaxy) was made and then the Klenow fragment was added, measurements being taken from time of adding the Klenow fragment.

Reaction Solution (Various Components Were Replaced with Water for the Control Reactions)

1.0 μl dNTP (without dATP, 25 mM of each type) 0.25 mM final

10 μl 10× Klenow buffer

72.5 μl ddH2O

0.5 μl dATP-Fluoresceine (0.05 mM) 0.25 μM final

4 μl Primer (12.5 pmol/μl) 0.5 pmol/μl final

10 μl PCR product (˜0.01-0.1 pmol/μl, with unknown amount of ssDNA)

1.0 μl Klenow (10 units/μl) 0.1 unit/μl final

Primer: CAGGAAACAGCTATGACACAATAAAACCATCCCAAATAC

The incubation temperature was maintained at 37° C.

Reaction Solutions:

| | B1 | B2 | B3 | B4 | B5 |
|---|---|---|---|---|---|
| master- | dNTP 0.25 mM | dNTP 0.25 mM | dNTP 0.25 mM | dNTP 0.25 mM | dNTP 0.25 mM |
| mix | 1 × Klenow | 1 × Klenow | 1 × Klenow | 1 × Klenow | 1 × Klenow |
| | buffer | buffer | buffer | buffer | buffer |
| | dATP-Fluo | dATP-Fluo | dATP-Fluo | dATP-Fluo | dATP-Fluo |
| | 0.25 μM | 0.25 μM | 0.25 μM | 0.25 μM | 0.25 μM |
| PCR | 10 μl | / | 10 μl | 10 μl | 5 μl |
| product | | | | | |
| Primer | 0.5 pmol/μl | 0.5 pmol/μl | / | 0.5 pmol/μl | 0.5 pmol/μl |
| (R74) | | | | | |
| Klenow | 0.1 unit/μl | 0.1 unit/μl | 0.1 unit/μl | / | 0.1 unit/μl |

Solutions B2, B3 and B4 were controls. Solution B1 is the reaction mixture, it showed a significant increase in floresence polarisation as illustrated in FIG. 3.

Primer Extension on a Solid Phase

The following reactions were carried out in a 384 microplate (black). The incorporation of dATP during the extension was monitored by measuring of the change of fluorescence polarisation.

4 different reaction solutions were compared. The mastermix was prepared with water, dNTPs, buffer and dATP-Fluoresceine and DNA (asymmetrc PCR product as above). The reaction mixtures were then completed according to the table below. Then the Gain Adjustment was made. The reaction was started by adding the Klenow fragment. Each minute the fluorescence polarisation was measured with the PolarStar for one hour.

Conditions for a 20 μl Prep.:

PCR product and

10 μl PCR product

2 μl 10× buffer

0.2 μl dNTP (dCTP, dGTP, dTTP) 0.25 mM final each

6.8 μl ddH2O

0.2 μl dATP-Fluoresceine (0.05 mM) 0.5 μM final

a) 1.6 μl Primer (12.5 pmol/μl) 0.5 pmol/μl final or b) bead bound primers (5 beads)

0.2 μl Klenow (10 units/μl) 0.1 unit/μl final

Incubattion temperature was constant at 37° C.

Primer:

| primer: | | | |
|---|---|---|---|
| free | R74 | ER1-B-L-M13b | CAGGAAACAGCTATGACACAATAAAACCATCCCAAATAC |
| bead | | | |
| bound | R78 | ER1-B-L-M13b-A | CAGGAAACAGCTATGACACAATAAAACCATCCCAAATAC |
| bead | | TNF-beta-L | |
| bound | R92 | M13b-A | CAGGAAACAGCTATGACAAAAACCCCAAAATAAACAA |

Template:

purified asymmetric PCR product from 18.11.01, tube 3b, 4b, 6b, pooled.

Wells:

20 μl preparation

| | 1 | 2 | 3 | 4 | |
|---|---|---|---|---|---|
| master- | dNTP 0.25 mM | dNTP 0.25 mM | dNTP 0.25 mM | dNTP 0.25 mM | blank |
| mix | 1 × Klenow | 1 × Klenow | 1 × Klenow | 1 × Klenow | 5 beads |
| | buffer | buffer | buffer | buffer | water |
| | dATP-Fluo | dATP-Fluo | dATP-Fluo | dATP-Fluo | |
| | 0.5 μM | 0.5 μM | 0.5 μM | 0.5 μM PCR | |
| | PCR product | PCR product | PCR product | product | |
| | 10 μl | 10 μl | 10 μl | 10 μl | |
| Primer | 1 pmol/μl | bead | bead | no primer | |
| | R74 | bound | bound | | |
| | ER1-B-L- | primers | primers | | |
| | M13b | R78 | R92 | | |
| | | ER1-B-L- | TNF-B-L- | | |
| | | M13b | M13b | | |
| Klenow | 0.1 unit/μl | 0.1 unit/μl | 0.1 unit/μl | 0.1 unit/μl | |
| (start | | | | | |
| reaction) | | | | | |

As can be seen from FIG. 4 an increase in fluorescence polarisation was observed in reaction mixtures 1, 2 and 3.

1. A method for the analysis of the methylation of cytosine bases in genomic DNA samples, comprising the following steps:
(a) the genomic DNA is chemically treated in such a manner that cytosine is converted into uracil or a similar base regarding the base pairing behaviour in the DNA duplex, 5 methylcytosine however remains unchanged;
(b) the chemically treated DNA is amplified using of at least one species of oligonucleotide (type A) as a primer in a polymerase reaction;
(c) the amplificate is left in solution with one or more species of fluorophore labelled nucleotides and one or more species of oligonucleotide (type B), wherein the type B oligonucleotide hybridises under appropriate conditions with its 3′ end directly on or up to 10 bases from the position to be examined, and wherein said type B oligonucleotide is at least partly nuclease resistant;
(d) the hybridised oligonucleotide (type B) is extended by means of a polymerase by at least one nucleotide, whereby the extension is dependant upon the methylation status of the respective cytosine position in the genomic DNA sample;
(e) the solution is incubated with a phosphodiesterase, which is capable of digesting nucleic acids, however incompletely digests the type B oligonucleotides and its extension products;
(f) the fluorescence polarisation of the solution is measured whereby for each fluorescent label used one determines the degree of polarisation.

2. A method according to claim 1 wherein all or a variable proportion of the fluorophore labelled nucleotides are dideoxynucleotides.

3. A method according to claims 1 and 2 whereby after the polymerase amplification of the bisulfite DNA the nucleotides of the polymerase reaction are diminished by means of a phosphatase and the phosphatase is subsequently thermally denatured.

4. A method according to claims 1 and 2 wherein the fluorescence polarisation of the fluorophore labelled nucleotides and/or dideoxynucleotides is measured prior to incorporation into the DNA duplex and again after incorporation into the DNA duplex.

5. A method according to claim 4 whereby the primer extension is detected by an increase in fluorescence polarisation.

6. A method according to claims 1 and 2 wherein said fluorophore is selected from the group consisting of 5′carboxyfluorescein, 6-carboxy-X-rhodamine, N,N,N′,N′,-tetramethyl-6-carboxy-X-rhodamine, BODIPY, Texas Red, Cy3, Cy5, FITC, DAPI, HEX, and TET.

7. A method according to claims 1 and 2 whereby the DNA sample is cleaved prior to bisulfite treatment with restriction endonucleases.

8. A method according to claims 1 and 2 whereby the DNA sample is isolated from human sources e.g. cell lines, blood, sputum, faeces, urine, brain, cerebrospinal fluid, tissue embedded in paraffin, for example tissue of eyes, intestine, kidney, brain, heart, prostate, lung, chest or liver, histological slides and all possible combinations.

9. A method according to claims 1 and 2 wherein the fluorescence polarisation of the enzymatically amplified DNA is measured directly from the container in which the polymerase reaction was carried out.

10. A method according to claims 1 and 2 wherein the Type B primers are immobilised on a surface prior to hybridisation with the amplificate.

11. A method according to claims 1 and 2 wherein the bisulfite treated DNA is immobilised on a surface prior to hybridisation with the fluorophore labelled nucleotides.

12. A method according to claims 10 and 11 whereby the surface comprises silicon, glass, polystyrene, aluminium, steel, iron, copper, nickel, silver or gold.

13. A method according to claims 1 and 2 whereby the information generated about the methylation status at the target site is provided to a computing device comprising one or more databases.

14. A method according to claims 1 and 2 whereby the information generated about the methylation status at the target site is provided to a computing device comprising one or more learning algorithms.

15. A diagnostic kit comprising:
a) one or more oligonucleotide primers designed to hybridise to bisulphite treated DNA sequence within 1-10 bases 3′ of the target site;
b) at least one species of nucleotides, wherein each species of nucleotide is covalently linked to a unique fluorophore;
c) DNA polymerase that reacts with the oligonucleotide primer and nucleotides to produce a 3′ extension of the primer.

16. A kit as in claim 15 whereby all or a variable proportion of the fluorophore linked nucleotides are in the form of dideoxynucleotides.


SEQUENCE LISTING (4 sequences)

SEQ ID NO: 1 - DNA, 20 bp, Artificial Sequence
  Primer Oligonucleotide
aggagggggaattaaataga

SEQ ID NO: 2 - DNA, 22 bp, Artificial Sequence
  Primer Oligonucleotide
acaataaaaccatcccaaatac

SEQ ID NO: 3 - DNA, 39 bp, Artificial Sequence
  Primer Oligonucleotide
caggaaacagctatgacacaataaaaccatcccaaatac

SEQ ID NO: 4 - DNA, 37 bp, Artificial Sequence
  Primer Oligonucleotide
caggaaacagctatgacaaaaaccccaaaataaacaa